(19) United States
(12) Patent Application Publication
(10) Pub. No.: US 2005/0112182 A1
(43) Pub. Date: May 26, 2005

(54) Medical adhesive and medical covering agent using ultraviolet ray curable chitosan derivative

(76) Inventors: Saburo Minami, Tottori-shi (JP); Eiko Renbutsu, Tottori-shi (JP); Yoshihiko Omura, Tottori-shi (JP); Fumiaki Nakatsubo, Uji-shi (JP); Yoshiharu Okamoto, Tottori-shi (JP); Hiroyuki Saimoto, Tottori-shi (JP); Yoshihro Shigemasa, Tottori-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/854,336

(22) Filed: May 27, 2004

(30) Foreign Application Priority Data
Nov. 20, 2003 (JP) 2003-391221

(51) Int. Cl. C08B 37/08; A61F 13/02; A61L 15/16

(52) U.S. Cl. 424/448; 536/20

(57) ABSTRACT

Provided is an N-alkyl chitosan derivative having an ultraviolet ray-curable functional group represented by formula (I) given below: 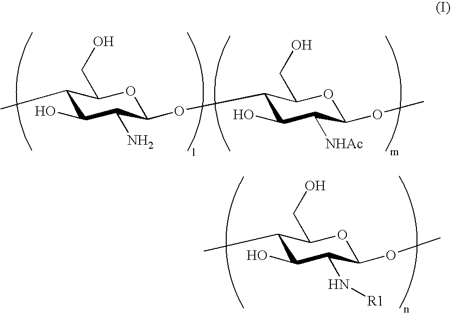
where l+m+n=1, 0<l<1, 0<m<0.5, 0<n<1 and R1 is represented by formula (II) given below: 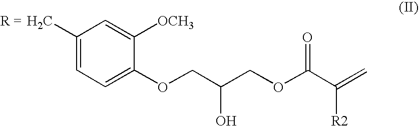 where R2 denotes a hydrogen atom or a methyl group.

### CROSS-REFERENCE TO RELATED APPLICATIONS

This application is based upon and claims the benefit of priority from prior Japanese Patent Application No. 2003-391221, filed Nov. 20, 2003, the entire contents of which are incorporated herein by reference.

### BACKGROUND OF THE INVENTION

1. Field of the Invention

The present invention relates to a medical adhesive and a medical covering agent used in a living body

2. Description of the Related Art

Conventional adhesives used for an organism can be roughly classified into a cyano acrylate series adhesive, a gelatin-aldehyde series adhesive, and a fibrin glue series adhesive. The cyano acrylate series adhesive and the gelatin-aldehyde series adhesive exhibit serious toxicity to the organism and impair the healing of a wound. On the other hand, it is possible for the fibrin glue series adhesive, which is low in its toxicity, to provide a culture medium of bacteria and, thus, to give rise to a danger of infection. In addition, a long time is required for the preparation of the fibrin glue series adhesive, with the result that the operating time is rendered long.

### BRIEF SUMMARY OF THE INVENTION

An object of the present invention, which has been achieved in view of the situation described above, is to provide a medical adhesive low in toxicity, excellent in adaptability to the organism, and easy to be handled, and to provide a medical covering agent excellent in its adaptability to the organism.

According to an aspect of the present invention, there is provided an N-alkyl chitosan derivative having an ultraviolet ray-curable functional group represented by general formula (I) given below:

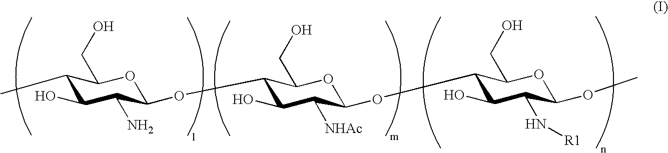

where l+m+n=1, 0<l<1, 0<m<0.5, 0<n<1 and R1 is represented by formula (II) given below:

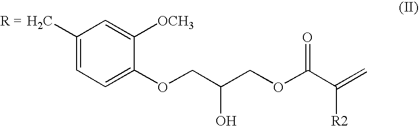

where R2 denotes a hydrogen atom or a methyl group.

It is preferable for the ranges of l, m and n given in formula (I) to be 0.1<l<1, 0<m<0.3, 0.2<n≦0.8, and most preferably the range of n should be 0.5<n≦0.8.

According to another aspect of the present invention, there is provided a medical adhesive containing an N-alkyl chitosan derivative having the ultraviolet ray-curable functional group defined above.

According to another aspect of the present invention, there is provided a medical covering agent containing an N-alkyl chitosan derivative having the ultraviolet ray-curable functional group defined above. The medical covering agent should preferably be a wound protective agent, a disinfectant having an adhesivity, a sealer at the injecting portion of a blood vessel catheter and a sealer for teat milk orifice or streak canal.

Further, according to still another aspect of the present invention, there is provided a medical adhesive low in toxicity, excellent in adaptability to the organism, and easy to be handled, and to provide a medical covering agent capable of preventing the infection by, for example, bacteria.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the preferred embodiments given below, serve to explain the principles of the invention.

FIG. 1 shows an FT/IR spectrum of an ultraviolet ray-curable chitosan derivative (I);

FIG. 2 is a photo showing the subcutaneous tissue at the injecting portion of dimethyl sulfoxide;

FIG. 3 is a photo showing the subcutaneous tissue at the injecting portion of an adhesive (which is not irradiated with ultraviolet rays) according to the present invention; and

FIG. 4 is a photo showing the subcutaneous tissue at the injecting portion of an adhesive (which is irradiated with ultraviolet rays) according to the present invention.

### DETAILED DESCRIPTION OF THE INVENTION

(1) Ultraviolet Ray-Curable Chitosan Derivative (I):

An N-alkyl chitosan derivative (I) having an ultraviolet ray-curable functional group defined in the present invention (hereinafter referred to as “ultraviolet ray-curable chitosan derivative (I)”) is capable of forming a polymer upon irradiation with ultraviolet rays so as to permit the tissue to be bonded to or closed. It follows that the ultraviolet ray-curable chitosan derivative (I) of the present invention can be used as an adhesive or, if formed in the form of a film, as a covering agent.

The ultraviolet ray-curable chitosan derivative (I) of the present invention clearly brought about a refuse reaction within an organism without irradiation with ultraviolet rays. However, it has been clarified that the polymer obtained after irradiation of the ultraviolet ray-curable chitosan derivative (I) with ultraviolet rays exhibits an adaptability to an organism.

Such being the situation, the ultraviolet ray-curable chitosan derivative (I) of the present invention can be used as an adhesive and a covering agent that can be applied to an organism in the medical field.

(2) Chemical Structure of Ultraviolet Ray-Curable Chitosan Derivative (I):

The ultraviolet ray-curable chitosan derivative (I) of the present invention can be represented by formula (I) given below: 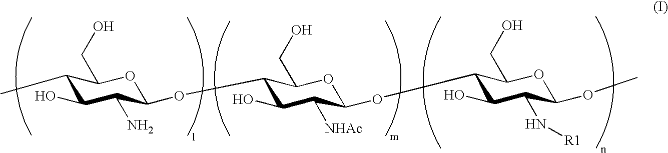
where R1 denotes an ultraviolet ray-curable functional group represented by formula (II) given below:

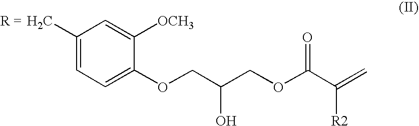

In formula (II) given above, R2 denotes a hydrogen atom or a methyl group, preferably a methyl group.

An N-alkyl chitosan derivative having an ultraviolet ray-curable functional group is disclosed in Japanese Patent Disclosure (Kokai) No. 2000-109501. The present inventors have made it possible to handle easily the ultraviolet ray-curable chitosan derivative (I) and to lower further the toxicity of the ultraviolet ray-curable chitosan derivative (I) by introducing an acetyl group into an N-alkyl chitosan derivative having an ultraviolet ray-curable functional group.

In chemical formula (I) given above, n denotes the number of N-alkyl groups substituted in the ultraviolet ray-curable chitosan derivative (I), and m denotes the number of acetyl groups substituted in the ultraviolet ray-curable chitosan derivative (I). It should be noted that, in formula (I), l+m+n should be 1, and the ranges of l, m, n should be 0<l<1, 0<m<0.5 and 0<n<1. It is more preferable for the ranges of l, m and n to be 0.1<l<1, 0<m<0.3, 0.2<n≦0.8, and most preferably the range of n should be 0.5<n≦0.8.

It has been found that the toxicity of the ultraviolet ray-curable chitosan derivative (I) is lowered with increase in the number of acetyl groups (i.e. m) substituted in the derivative (I). On the other hand, if the number m of acetyl groups noted above is increased, the insolubility of the derivative (I) is increased so as to make it difficult to introduce the functional group into the derivative (I), with the result that it is difficult to polymerize the derivative (I) (data not shown).

(3) Method of Synthesizing Ultraviolet Ray-Curable Chitosan Derivative (I):

The ultraviolet ray-curable-chitosan derivative (I) of the present invention can be obtained by the reaction between chitosan having a desired degree of deacetylation and a (meth)acrylic acid derivative having an aldehyde group.

It is possible to obtain chitosan having a desired degree of deacetylation by deacetylating chitin. Also, chitosan having a desired degree of deacetylation, which can be used in the present invention, is available on the market. The molecular weight of chitosan is preferable in the range of about 1000-2000000, in particular the range of about 5000-150000.

The (meth)acrylic acid derivative having an aldehyde group, which can be used in the present invention, is not particularly limited as far as the derivative has an aldehyde group capable of reaction with the amino group of chitosan and also has an acryloyl group or a methacryloyl group that is cured upon irradiation with ultraviolet light. It is preferable to use an aromatic aldehyde having an acryloyl group or a methacryloyl group including, for example, 2-hydroxy-3-(4-formyl-2-methoxy)phenoxy propyl acrylate and 2-hydroxy-3-(4-formyl-2-methoxy)phenoxy propyl methacrylate. The particular acrylic acid derivative can be synthesized by the method referred to in the Examples described herein later. It is also possible to synthesize the acrylic acid derivative used in the present invention by a known method disclosed in, for example, “J. Polym. S., Part A: Polym. Chem., Vol. 25,3063-3077 (1987)”.

For carrying out the reaction between chitosan and a (meth)acrylic acid derivative, chitosan having a desired degree of deacetylation is dissolved first in an dilute organic acid solution such as formic acid or acetic acid. Then, after a hydrophilic solvent such as methanol is added to the resultant solution, a solution of (meth)acrylic acid derivative is added to the solution so as to prepare a reaction system. The reaction system thus prepared is stirred for 6 to 12 hours. Further, a solution of a reducing agent such as sodium borohydride is added to the reaction system, and the reaction system is stirred for 6 to 12 hours.

The ratio in amount of chitosan to (meth)acrylic derivative can be determined appropriately in view of the desired degree of substitution of the N-alkyl groups. Also, the reaction temperature can be in the range of between 0° C. and room temperature.

The reaction product can be identified by an infrared absorption spectrum and a proton nuclear magnetic resonance spectrum.

In synthesizing ultraviolet ray-curable chitosan. derivative (I) in which R2 included in formula (II) denotes a hydrogen atom, 2-hydroxy-3-(4-formyl-2-methoxy)phenoxy propyl acrylate is used as the (meth)acrylic acid derivative having an aldehyde group. Also, 2-hydroxy-3-(4-formyl-2-methoxy)phenoxy propyl methacrylate (VMA) is used for synthesizing ultraviolet ray-curable chitosan derivative (I) in which R2 noted above denotes a methyl group.

(4) Medical Adhesive using Ultraviolet Ray-Curable Chitosan Derivative (I):

For preparing the medical adhesive according to the present invention, the ultraviolet ray-curable chitosan derivative (I) is mixed with solvents such as dimethyl sulfoxide and water, and a photopolymerization initiator is added to the mixture. It is possible to use buffers such as a phosphate buffer and an acetate buffer or a physiological saline in place of water.

It is also possible to add another ultraviolet ray-curable compound. The kind, the addition amount, etc. of the ultraviolet ray-curable compound can be determined appropriately in view of the object and the use of the compound.

The resultant adhesive mixture is generally a liquid, which is injected by using, for example, a syringe into the portion where the liquid adhesive is to be used. Then, the injected adhesive is irradiated with ultraviolet rays for curing the adhesive. The irradiation with the ultraviolet rays can be performed under conditions employed in the known method.

It is possible to use a known polymerization initiator such as benzophenone, benzoin, acetophenone, benzoin methyl ether, benzoin ethyl ether, and methyl benzoyl formate.

The adhesive of the present invention, which exhibits an adaptability to an organism, can be used for bonding tissues in performing a surgical operation and can also be used as an adhesive for bonding the tissues of the skin, the blood vessel, the internal organs, etc.

(5) Medical Covering Agent Using Ultraviolet Ray-Curable Chitosan Derivative (I):

The medical covering agent of the present invention is similar in construction to the adhesive described above. It can be coated to the diseased portion or the like, followed by irradiation with ultraviolet rays, thereby it can be used as a film-like polymer. By forming the polymer into a film, it is possible to use the polymer film as a covering agent capable of preventing the infection of bacteria or the like and exhibiting adhesivity. It should also be noted that the medical covering agent of the present invention is low in its toxicity and does not impair the healing of a wound and, thus, can be used as a wound protective agent. In addition, it is possible to add a medicine exhibiting a sterilizing function to the medical covering agent so as to provide a disinfectant. Further, the medical covering agent of the present invention can be used as a sealer at the injecting portion of a blood vessel catheter. Still further, the medical covering agent of the present invention can be applied to a surgical drape so as to coat the surface of the base material with the medical covering agent.

The medical covering agent of the present invention can also be used as a covering agent for sealing the teat milk orifice or streak canal of a cow so as to prevent the entry of bacteria.

The medical adhesive or covering agent of the present invention does not provide a culture medium of bacteria and, thus, is free from the danger of infection. Also, since the polymerization is initiated upon irradiation with light, it is unnecessary to mix a plurality of medications. It follows that the medical adhesive or covering agent of the present invention can be handled easily.

### EXAMPLES

Some Examples of the present invention will now be described.

### Example 1

### Synthesis of VMA

The synthetic reaction scheme of 2-hydroxy-3-(4-formyl-2-methoxy)phenoxy propyl methacrylate (VMA), which is also called 3-methoxy-4-(2-hydroxy-3-methacryloyloxy propoxy)benzaldehyde and which is one of ultraviolet ray-curable functional groups constituting the ultraviolet ray-curable chitosan derivative (I) of the present invention, is as shown below:

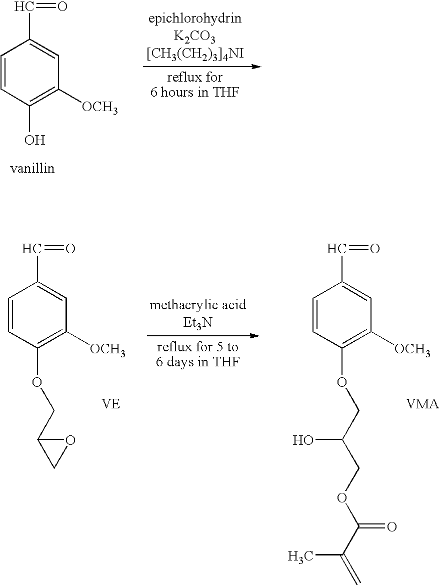

7.6 g (50 mmol) of vanillin, 8.3 g (60 mmol) of potassium carbonate, a catalytic amount of quaternary ammonium salt, e.g., tetra-n-butyl ammonium iodide, and 40 ml of epichlorohydrin were suspended in 120 ml of tetrahydrofuran (THF), and the suspension thus obtained was put under reflux at 90° C. for 6 hours. After being condensed, the suspension was washed with water, and purified with silica gel column chromatography. Further, the purified material was crystallized in ethanol so as to obtain 6.5 g of pale yellow needle crystals of a vanillin derivative (VE). The yield of the needle crystals was 62%.

Five grams of the vanillin derivative (VE) crystals, which was taken from the needle crystals noted above, was dissolved in 100 ml of THF together with 2.5 ml (29.5 mmol) of methacrylic acid, 1 ml (7 mmol) of triethyl amine and 400 mg (3.2 mmol) of hydroquinone monomethyl ether, and the resultant solution was put under reflux at 90° C. for 6 days. After being condensed, the solution was washed with water and, then, purified with silica gel column. chromatography. Further, the purified material was crystallized by using ethyl acetate so as to obtain 4.6 g of pale yellow needle crystals of the aimed product (VMA). The yield of the product was 65.6%.

The result of the mass analysis was 294.11 (molecular weight 294.30, C₁₅H₁₈O₆), and the result of the element analysis was C, 61.22; H, 6.16; O, 32.62(%).

### Example 2

### Synthesis of Ultraviolet Ray-Curable Chitosan Derivative

1.66 g of Dytoxane FP-1 (available from Dai-nichi Seika Kogyo K.K., having a molecular weight of 20,000 to 30,000 and having a deacetylation degree of 97) was dissolved in 100 ml of acetate buffer adjusted at a pH value of 4.5 and, then, diluted with methanol.

The solution thus obtained was cooled with an ice bath, followed by dripping a methanol solution containing 2.35 g of VMA onto the cooled solution. After stirring overnight at room temperature, the reacting solution was cooled again with an ice bath and, then, an aqueous solution having 661 mg of sodium cyano borohydride dissolved therein was dripped into the reacting solution. After reaction for one hour within the ice bath and after reaction overnight at room temperature, the reacting solution was neutralized with 1% of sodium hydroxide, followed by dialysis with distilled water for one week.

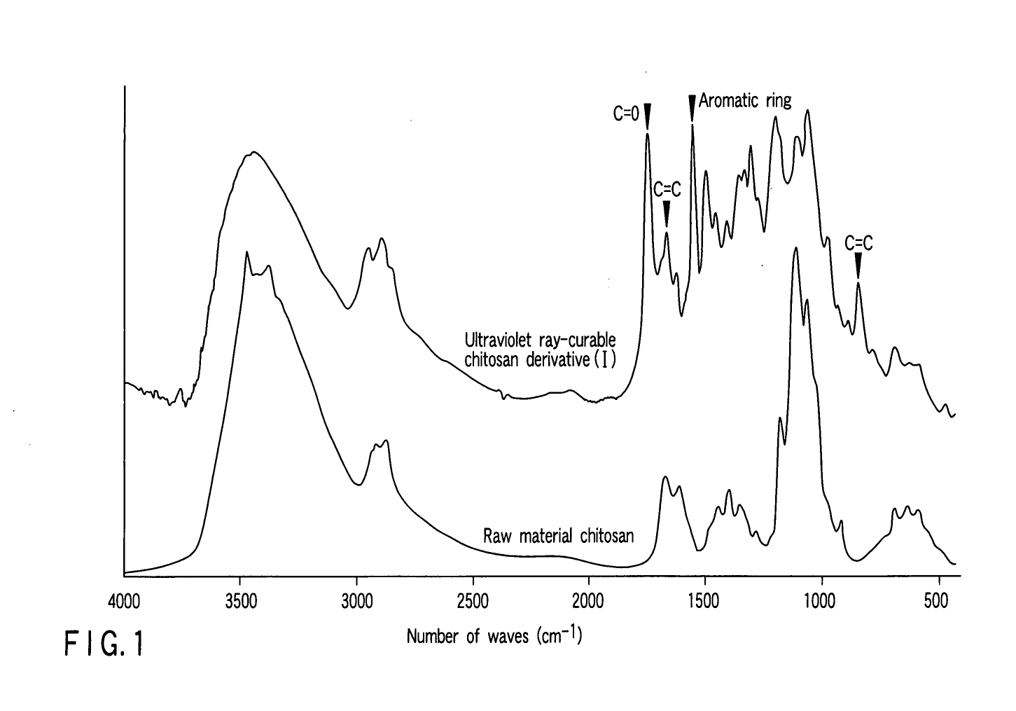

The formed product within the dialysis tube was collected by centrifugal separation so as to obtain a hydrous paste containing about 3% by dry weight of ultraviolet ray-curable chitosan derivative (I). FIG. 1 shows the result of the FT/IR spectrum of the formed product. The size of the absorption peak derived from the side chain, which is shown in the drawing, indicates that the degree of substitution of the ultraviolet ray-curable functional group was 0.7 to 0.8.

### Example 3

### Test for Examining the Adaptability to Organism of the Medical Adhesive Using Ultraviolet Ray-Curable Chitosan Derivative (I))

The adaptability to an organism was tested in respect of the medical adhesive according to one embodiment of the present invention. The composition of the adhesive was as shown in Table 1.

TABLE 1

| Components | Amount | |
|---|---|---|
| Ultraviolet ray-curable | 5 | wt % |
| chitosan derivative (I); | | |
| Water | 11 | wt % |
| Dimethyl sulfoxide | 84 | Wt % |

| | |
|---|---|
| Photopolymerization | catalytic amount |
| initiator | |

Three incision each having a length of 2 cm in the direction of the body axis were formed on the back of a dog anesthetized with acepromazine (0.5 mg/Kg) and pentobarbital (25 mg/Kg), and each of the subcutaneous regions of the incision portions was enlarged with scissors so as to have a diameter of about 2 cm.

Three kinds of treatments (A), (B) and (C) given below were applied to each of the incision portions:

(A) Dimethyl sulfoxide used as a reference substance was injected in an amount of 0.5 ml, and irradiation with an ultraviolet ray was not performed.

(B) The adhesive was injected in an amount of 0.5 ml, and irradiation with an ultraviolet ray was not performed.

(C) The adhesive was injected in an amount of 0.5 ml, and irradiation with an ultraviolet ray was performed. The ultraviolet ray irradiation was performed under the conditions given in Table 2 below.

TABLE 2

| Item | Details and conditions |
|---|---|
| UV spot irradiating | Ex250 UV Light Source |
| machine | (HOYA Schott Co. Ltd.) |
| Lamp style | 250 DL (250 W) |
| Irradiating distance | 3 cm |
| Irradiating time | 2.5 seconds |
| Irradiating energy | 1625 mj/cm² |

After the treatments given above, each of the incision portions was sutured with an absorptive suturing string, and the pain (oppressive pain), feverishness, swelling, rubefaction, and general states were observed for one week in order to confirm the clinical inflammation reaction. One week later, each portion was collected under a general anesthesia so as to carry out the histological analysis. The result of the observation is shown in Table 3 below.

TABLE 3

| | | | | Clinical | |
|---|---|---|---|---|---|
| | | Histological analysis | | inflammation | |
| | | | Foreign | reaction (pain) | |
| Injected | UV | | body giant | Second | Third |
| material | irradiation | Fibroblast | cell | day | day |
| (A) reference | − | thick tissue hy- | confirmed | ± | − |
| | | perplasia around | | | |
| | | the injection | | | |
| | | cavity | | | |
| (B) adhesive | − | tissue hyperplasia | clearly | + | − |
| | | around the injec- | confirmed | | |
| | | tion cavity | | | |
| | | hyperplasia a- | | | |
| | | round the injected | | | |
| | | material | | | |
| (C) adhesive | + | tissue hyperplasia | not | + | − |
| | | around the injec- | confirmed | | |
| | | tion cavity | | | |
| | | tissue hyperplasia | | | |
| | | around and inside | | | |
| | | the injected | | | |
| | | material | | | |

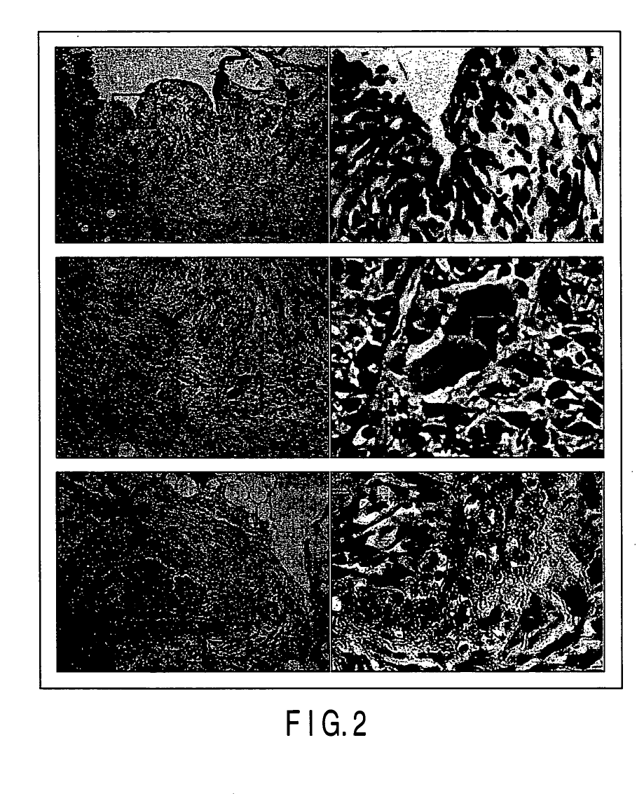

As shown in FIG. 2, the growth of fibroblast and red blood cell were recognized around the injected material in tissue (A) that treated the reference material. The fibroblast was activated, and a foreign body giant cell was also observed. Although the clinical inflammation reaction was not recognized, the appearance of the red blood cell was recognized around the nerve, supporting that the inflammation reaction was prominent histologically.

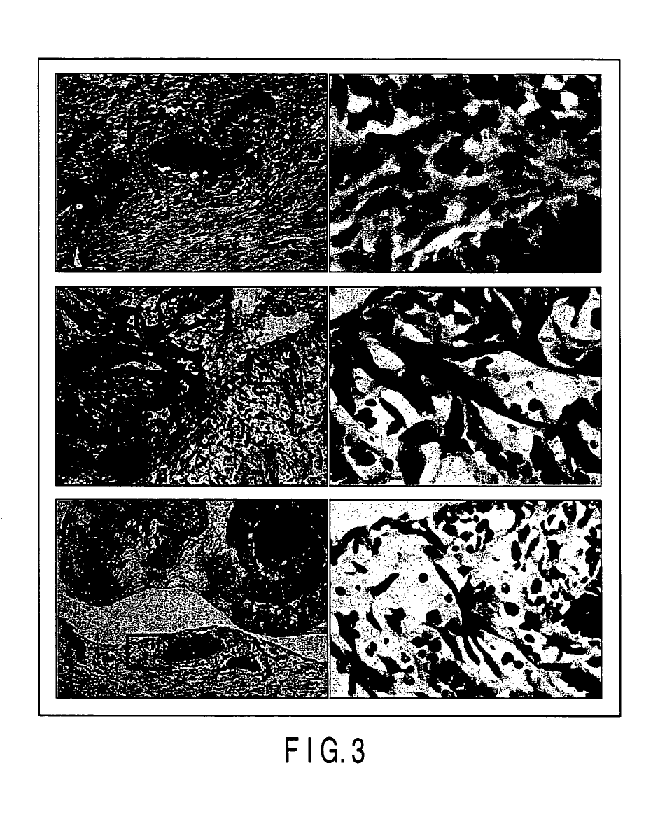

As shown in FIG. 3, the increasing of fibroblasts were recognized around the injecting cavity and around the injected material in the tissue (B) covering the case where the adhesive was injected and the adhesive was not irradiated with ultraviolet light. However, there was few migration of fibroblast inside the injected material. Also, a foreign body giant cell was clearly recognized. It follows that, in each of the reference substance and the adhesive that had not been irradiated with ultraviolet rays, the buried material was recognized as a foreign matter within the organism.

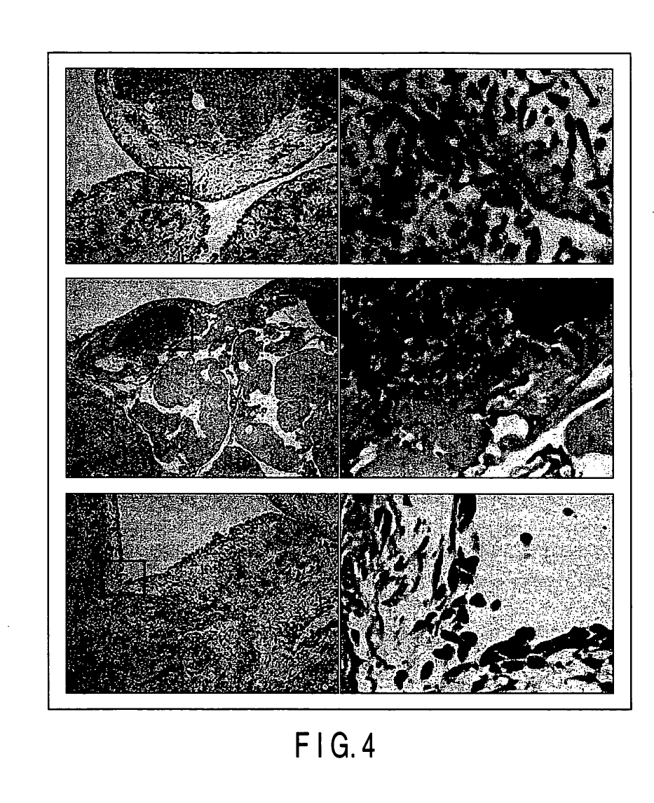

On the other hand, the increasing of fibroblast was recognized around and inside the injected material in adhesive (C) irradiated with ultraviolet rays, as shown in FIG. 4. However, a foreign body giant cell was not recognized. This indicates that the cell does not regard chitosan derivative (I) cured by the ultraviolet ray irradiation as foreign matter.

As described above, it has been clarified that the adhesive using the ultraviolet ray-curable chitosan derivative (I) of the present invention exhibits an adaptability to an organism after the ultraviolet ray-curable chitosan derivative (I) is cured by ultraviolet irradiation.

Additional advantages and modifications will readily occur to those skilled in the art. Therefore, the present invention in its broader aspects is not limited to the specific details and representative embodiments shown and described herein. Accordingly, various modifications may be made without departing from the spirit or scope of the general inventive concept as defined by the appended claims and their equivalents.

1. An N-alkyl chitosan derivative having an ultraviolet ray-curable functional group represented by formula (I) given below: 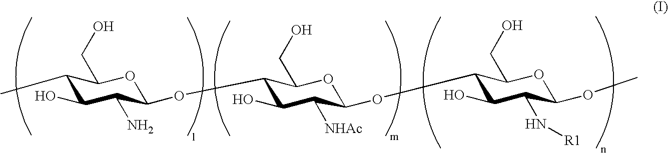
where l+m+n=1, 0<l<1, 0<m<0.5, 0<n<1 and R1 is represented by formula (II) given below: 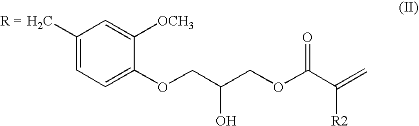
where R2 denotes a hydrogen atom or a methyl group.

2. The N-alkyl chitosan derivative according to claim 1, wherein the ranges of l, m and n given in formula (I) are 0.1<l<1, 0<m<0.3, 0.2<n≦0.8.

3. The N-alkyl chitosan derivative according to claim 2, wherein the range of n given in formula (I) is 0.5<n≦0.8.

4. A medical adhesive, comprising the N-alkyl chitosan derivative which having an ultraviolet ray-curable functional group represented by formula (I) given below: 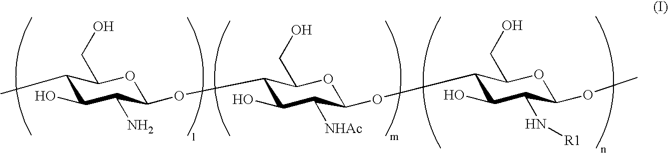
where l+m+n=1, 0<l<1, 0<m<0.5, 0<n<1 and R1 is represented by formula (II) given below: 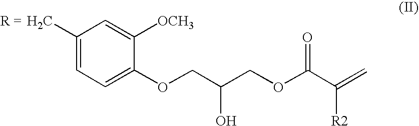
where R2 denotes a hydrogen atom or a methyl group.

5. The medical adhesive according to claim 4, wherein the ranges of l, m and n given in formula (I) are 0.1<l<1, 0<m<0.3, 0.2<n≦0.8.

6. The medical adhesive according to claim 5 wherein the range of n given in formula (I) is 0.5<n≦0.8.

7. A medical covering agent, comprising the N-alkyl chitosan derivative which having an ultraviolet ray-curable functional group represented by formula (I) given below: 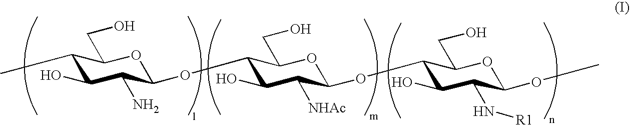
where l+m+n=1, 0<l<1, 0<m<0.5, 0<n<1 and R1 is represented by formula (II) given below: 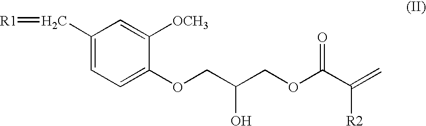
where R2 denotes a hydrogen atom or a methyl group.

8. The medical covering agent according to claim 7, wherein the ranges of l, m and n given in formula (I) are 0.1<l<1, 0<m<0.3, 0.2<n≦0.8.

9. The medical covering agent according to claim 8, wherein the range of n given in formula (I) is 0.5<n≦0.8.

10. The medical covering agent according to claim 7, wherein the medical covering agent is a wound protecting agent.

11. The medical covering agent according to claim 7, wherein the medical covering agent is a disinfectant exhibiting adhesivity.

12. The medical covering agent according to claim 7, wherein the medical covering agent is a sealer for the injecting portion of a blood vessel catheter.

13. The medical covering agent according to claim 7, wherein the medical covering agent is a sealer for the teat milk orifice and streak canal.
